(19) United States
(12) Patent Application Publication
(10) Pub. No.: US 2024/0404699 A1
(43) Pub. Date: Dec. 5, 2024

(54) MEDICAL INFORMATION PROCESSING APPARATUS, MEDICAL INFORMATION PROCESSING METHOD, AND STORAGE MEDIUM

(71) Applicant: CANON MEDICAL SYSTEMS CORPORATION, Tochigi (JP)

(72) Inventor: Yudai YAMAZAKI, Nasushiobara (JP)

(73) Assignee: CANON MEDICAL SYSTEMS CORPORATION, Tochigi (JP)

(21) Appl. No.: 18/678,178

(22) Filed: May 30, 2024

(30) Foreign Application Priority Data
May 30, 2023 (JP) 2023-088849

(51) Int. Cl. G16H 50/20; G16H 30/00; G16H 50/50

(52) U.S. Cl. CPC G16H 50/20; G16H 30/00; G16H 50/50

(57) ABSTRACT

A medical information processing apparatus according to an embodiment acquires a first medical data group and a second medical data group different from the first medical data group, determines a first position in a model manifold associated with a statistical model by projecting the first medical data group onto the model manifold, determines second positions in the model manifold by projecting the second medical data group onto the model manifold, and calculates a geodesic distance based on the first position and the second position.

### CROSS-REFERENCE TO RELATED APPLICATIONS

This application is based upon and claims the benefit of priority from Japanese Patent Application No. 2023-088849, filed on May 30, 2023; the entire contents of which are incorporated herein by reference.

### FIELD

Embodiments disclosed herein relate generally to a medical information processing apparatus, a medical information processing method, and a storage medium.

### BACKGROUND

In clinical settings, various medical data are acquired and recorded in databases. In addition to presenting these medical data to users such as physicians who make a diagnosis, recent years have seen the development of technologies that perform information processing on these medical data and provide diagnostic support based on the results of the information processing.

### BRIEF DESCRIPTION OF THE DRAWINGS

FIG. 1 is a block diagram illustrating an example of the configuration of a medical information processing system according to a first embodiment;

FIG. 2 is a diagram for explaining a method for calculating a distance between manifolds according to the first embodiment;

FIG. 3 is a schematic diagram illustrating a process of a medical information processing apparatus according to the first embodiment;

FIG. 4 is a diagram for explaining projection of a medical data group onto a model manifold according to the first embodiment;

FIG. 5 is a diagram for explaining calculation of a geodesic distance according to the first embodiment;

FIG. 6A is a diagram illustrating an example of a distance calculation result according to the first embodiment;

FIG. 6B is a diagram illustrating an example of a distance calculation result according to the first embodiment;

FIG. 6C is a diagram illustrating an example of a distance calculation result according to the first embodiment;

FIG. 7 is a diagram illustrating a display example according to the first embodiment;

FIG. 8A is a diagram illustrating a display example according to the first embodiment;

FIG. 8B is a diagram illustrating a display example according to the first embodiment; and

FIG. 9 is a flowchart illustrating an example of a process of the medical information processing apparatus according to the first embodiment.

### DETAILED DESCRIPTION

A medical information processing apparatus according to embodiments comprise processing circuitry configured to: acquire a first medical data group and a second medical data group different from the first medical data group; determine a first position in a model manifold associated with a statistical model by projecting the first medical data group onto the model manifold, and determine second positions in the model manifold by projecting the second medical data group onto the model manifold; and calculate a geodesic distance based on the first position and the second position.

Embodiments of a medical information processing apparatus, a medical information processing method, and a storage medium are described in detail below with reference to the accompanying drawings.

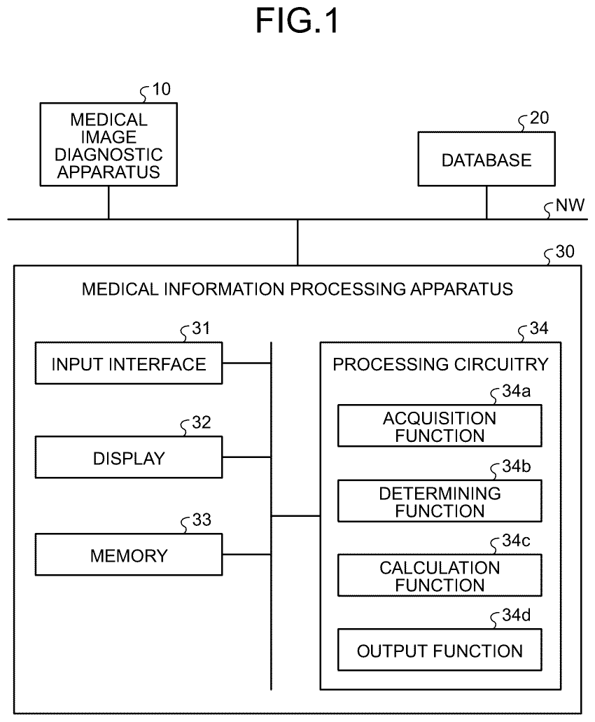

The first embodiment takes, as an example, a medical information processing system 1 including a medical information processing apparatus 30. For example, as illustrated in FIG. 1, the medical information processing system 1 includes a medical image diagnostic apparatus 10, a database 20, and the medical information processing apparatus 30. FIG. 1 is a block diagram illustrating an example of the configuration of the medical information processing system 1 according to the first embodiment.

As illustrated in FIG. 1, the medical image diagnostic apparatus 10, the database 20, and the medical information processing apparatus 30 are connected to one another via a network NW. The network NW may be configured as a local network closed within a hospital, or may be a network via the Internet. For example, the database 20 may be installed in the same facility as the medical image diagnostic apparatus 10 and the medical information processing apparatus 30, or may be installed in a different facility.

The medical image diagnostic apparatus 10 is an apparatus that acquires medical images from a subject. The type (modality) of the medical image diagnostic apparatus 10 is not particularly limited, but examples of the medical image diagnostic apparatus 10 may include an X-ray diagnostic apparatus, an X-ray computed tomography (CT) apparatus, a magnetic resonance imaging (MRI) apparatus, an ultrasound diagnostic apparatus, a single photon emission computed tomography (SPECT) apparatus, and a positron emission computed tomography (PET) apparatus. Although FIG. 1 illustrates a single medical image diagnostic apparatus 10, the medical information processing system 1 may include a plurality of medical image diagnostic apparatus 10. The medical image acquired by the medical image diagnostic apparatus 10 is an example of medical data.

The database 20 is a storage that stores various medical data, and is implemented, for example, by computer equipment such as a server and a workstation. The database 20 may be a server of an information management system such as a radiology information system (RIS), a hospital information system (HIS), and a picture archiving and communication system (PACS). Although FIG. 1 illustrates a single database 20, the database 20 may be implemented by a combination of a plurality of storages.

The medical information processing apparatus 30 enables a process by processing circuitry 34 to easily provide diagnostic support that comprehensively takes medical data into account. For example, as illustrated in FIG. 1, the medical information processing apparatus 30 includes an input interface 31, a display 32, a memory 33, and the processing circuitry 34.

The input interface 31 receives various input operations from a user, converts the received input operations into electrical signals, and outputs the electrical signals to the processing circuitry 34. For example, the input interface 31 is implemented by a mouse, a keyboard, a trackball, a switch, a button, a joystick, a touchpad for performing input operations by touching an operation surface, a touchscreen that integrates a display screen and a touchpad, non-contact input circuitry using an optical sensor, voice input circuitry, and the like. The input interface 31 may be configured as a tablet terminal or the like capable of wirelessly communicating with a body of the medical information processing apparatus 30. The input interface 31 may also be circuitry that receives input operations from the user through motion capture. To give an example, the input interface 31 can receive user's body movements, gaze, and the like as input operations by processing signals acquired via a tracker and images acquired about the user. The input interface 31 is not limited only to those with physical operating components such as a mouse and a keyboard. For example, an example of the input interface 31 also includes electrical signal processing circuitry that receives electrical signals corresponding to input operations from an external input device provided separately from the medical information processing apparatus 30 and transmits the electrical signals to the processing circuitry 34.

The display 32 displays various information. For example, the display 32 displays diagnostic support information under the control of the processing circuitry 34. For example, the display 32 displays a graphical user interface (GUI) for receiving various instructions, settings, and the like from the user via the input interface 31. For example, the display 32 is a liquid crystal display or a cathode ray tube (CRT) display. The display 32 may be of a desktop type, or may be configured as a tablet terminal or the like capable of wirelessly communicating with the body of the medical information processing apparatus 30.

In FIG. 1, although the medical information processing apparatus 30 is described as including the display 32, the medical information processing apparatus 30 may include a projector instead of or in addition to the display 32. The projector can perform projection onto a screen, a wall, a floor, or the like under the control of the processing circuitry 34. To give an example, the projector can also perform projection onto an arbitrary plane, object, space, or the like by projection mapping.

The memory 33 is implemented by a semiconductor memory element such as a random access memory (RAN) and a flash memory, a hard disk, an optical disk, or the like. For example, the memory 33 stores medical data acquired from the database 20 and computer programs for the circuitry included in the medical information processing apparatus 30 to implement functions thereof. The memory 33 may be implemented by a server group (cloud) connected to the medical information processing apparatus 30 via the network NW.

The processing circuitry 34 controls the operation of the entire medical information processing apparatus 30 by performing an acquisition function 34a, a determining function 34b, a calculation function 34c, and an output function 34d. The acquisition function 34a is an example of an acquisition unit. The determining function 34b is an example of a determining unit. The calculation function 34c is an example of a calculation unit. The output function 34d is an example of an output unit.

For example, the processing circuitry 34 reads a computer program corresponding to the acquisition function 34a from the memory 33 and executes the read computer program, thereby acquiring a first medical data group and a second medical data group different from the first medical data group. The processing circuitry 34 reads a computer program corresponding to the determining function 34b from the memory 33 and executes the read computer program, thereby determining a first position in a model manifold by projecting the first medical data group onto the model manifold and determining a second position in the model manifold by projecting the second medical data group onto the model manifold. The processing circuitry 34 reads a computer program corresponding to the calculation function 34c from the memory 33 and executes the read computer program, thereby calculating a geodesic distance based on the first position and the second position. The processing circuitry 34 reads a computer program corresponding to the output function 34d from the memory 33 and executes the read computer program, thereby providing output based on the geodesic distance calculated by the calculation function 34c.

The geodesic distance calculated by the calculation function 34c is used to generate diagnostic support information. The generation and display of the diagnostic support information may be performed by the medical information processing apparatus 30 or by other apparatuses. For example, the output function 34d generates the diagnostic support information on the basis of the geodesic distance, and displays the diagnostic support information on the display 32. Alternatively, the output function 34d generates the diagnostic support information on the basis of the geodesic distance, and transmits the generated diagnostic support information to other apparatus via the network NW. In this case, the other apparatus display the diagnosis support information. Alternatively, the output function 34d transmits the geodesic distance calculated by the calculation function 34c to other apparatus via the network NW. In this case, the other apparatus generates and displays the diagnosis support information. Details of the processes by the acquisition function 34a, the determining function 34b, the calculation function 34c, and the output function 34d are described below.

In the medical information processing apparatus 30 illustrated in FIG. 1, each processing function is stored in the memory 33 in the form of a computer program executable by a computer. The processing circuitry 34 is a processor that reads the computer programs from the memory 33 and executes the read computer programs, thereby implementing functions corresponding to the executed computer programs. In other words, the processing circuitry 34 in the state of reading the computer programs has functions corresponding to the read computer programs.

In FIG. 1, the acquisition function 34a, the determining function 34b, the calculation function 34c, and the output function 34d are described as being implemented by the single processing circuitry 34; however, the processing circuitry 34 may be configured by combining a plurality of independent processors, and the processors may implement respective functions by executing computer programs. The processing functions of the processing circuitry 34 may be implemented by being appropriately distributed or integrated into single processing circuitry or a plurality of pieces of processing circuitry.

The processing circuitry 34 may also implement the functions by using a processor of an external apparatus connected via the network NW. For example, the processing circuitry 34 reads computer programs corresponding to the respective functions from the memory 33 and executes the read computer programs, and implement the respective functions illustrated in FIG. 1 by using a server group (cloud) connected to the medical information processing apparatus 30 via the network NW as computing resources.

The medical information processing system 1 including the medical image diagnostic apparatus 10, the database 20, and the medical information processing apparatus 30 has been described above. Under such a configuration, the medical information processing apparatus 30 can easily provide diagnostic support that comprehensively takes medical data into account.

A method of diagnostic support is to allow a machine learning model to generate diagnostic support information. In this case, learning data is first acquired and a machine learning model is built to solve a specific task. The learning data is generally acquired by disease, department, and modality.

For example, a machine learning model that provides diagnostic support for the disease “lung cancer” can be generated using X-ray CT images as learning data. To give an example, a machine learning model functionalized to determine the presence or absence of lung cancer can be generated by training a neural network using X-ray CT images as input-side data and using a confirmed diagnosis of whether a subject targeted for each X-ray CT image is suffering from lung cancer as output-side data. Subsequently, the machine learning model can receive the X-ray CT image and output the result of determining the presence or absence of lung cancer as diagnostic support information.

However, such machine learning-based diagnostic support needs to prepare tasks and learning data in advance. Acquiring sufficient learning data may not be easy and may not be able to respond to changes in data trends or tasks. For example, a change in disease classification requires relearning.

Another method of diagnostic support based on medical data is to determine subjects with similar medical data. For example, medical data of a subject to be diagnosed can be compared with medical data of other subjects to determine subjects with similar medical data, and from the medical data of the determined subjects, and the condition of the subject to be diagnosed and lesions that the subject may suffer from can be estimated and provided to a user as diagnostic support information.

However, various medical data are acquired in clinical settings. For example, subject information such as the height, weight, and medical history of a subject, medical images taken by the medical image diagnostic apparatus 10, and test results such as the pulse rate, electrocardiogram, and blood gas readings of the subject are acquired as medical data and registered in the database 20. While it is easy to determine subjects who are similar only with respect to specific items, for example, subjects with a similar height, it is not easy to determine subjects who are similar overall with respect to various medical data.

A mutual subspace method is known as one of methods for calculating the distance (similarity) of medical data between subjects. In the mutual subspace method, a subspace approximating each data is calculated, and the proximity of angles between the subspaces is calculated as the similarity of the data. However, since the mutual subspace method assumes linearity, the accuracy is reduced when the distribution of medical data is nonlinear.

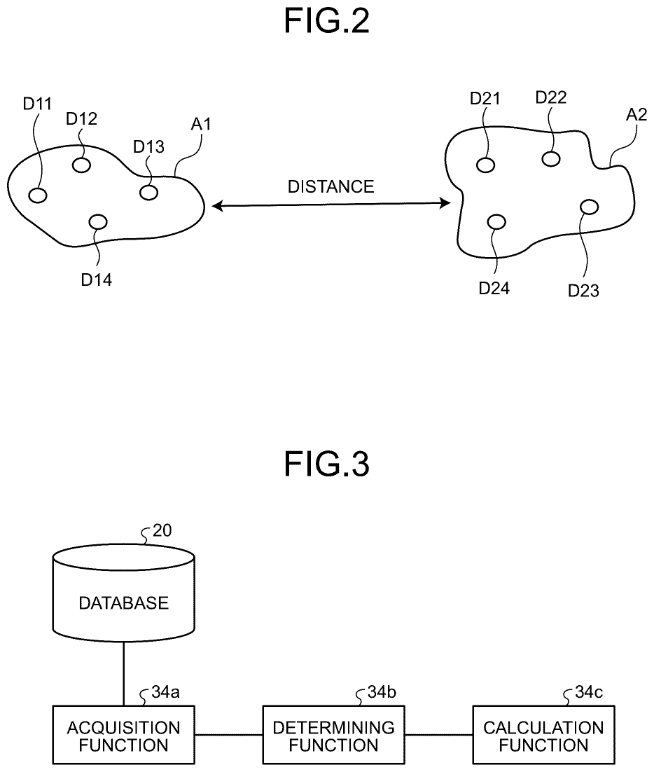

A method for calculating a distance between manifolds has been proposed as a way to maintain accuracy even when targeting non-linear medical data. That is, as illustrated in FIG. 2, for example, various medical data of a subject are captured as manifolds, and a distance between the manifolds is calculated as the similarity. FIG. 2 illustrates a manifold A1 including data D11, data D12, data D13, and data D14 and a manifold A2 including data D21, data D22, data D23, and data D24. The data D11 to D14 are, for example, various medical data acquired on a specific subject, and the data D21 to D24 are various medical data acquired on the subject different from that of the data D11 to D14. A manifold including medical data is also described as a biological manifold.

However, a manifold is a space with an expanse as illustrated in FIG. 2, and when calculating a distance in a data space, for example, representative points established for the respective manifolds are compared, and an entire data distribution included in the manifold is not evaluatable. That is, diagnostic support that comprehensively takes available medical data into account is not able to be provided. For example, when the distance is calculated using medical data for each subject as a manifold, only the instantaneous condition of each subject (for example, when the subject is in good or bad physical condition) can be captured, and the constant condition of the subject is not evaluatable.

In this regard, the medical information processing apparatus 30 can easily provide diagnostic support that comprehensively takes medical data into account through the following process. Details of the process are described below with reference to the schematic diagram in FIG. 3.

Various medical data are registered in advance in the database 20 in FIG. 3. The medical data is sequentially stored in the database 20, including while the process to be described below is being performed by the medical information processing apparatus 30.

Examples of the medical data include subject information (patient information). The subject information is, for example, information on a subject such as a patient ID, name, date of birth, gender, blood type, height, and weight. For example, the subject information is registered in a system such as HIS or RIS as a result of a medical interview when the subject visits a hospital.

Examples of the medical data also include medical images taken by the medical image diagnostic apparatus 10. For example, various medical images are acquired by various modalities installed in the hospital and registered in a system such as PACS. The database 20 may also record measurements based on medical images as medical data. For example, the database 20 records medical data such as a blood vessel diameter measured on the basis of an angiographic X-ray image and a blood flow velocity measured on the basis of an ultrasound image.

Examples of the medical data also include test results from tests such as blood tests, biochemical tests, and vital sign measurements. For example, test information is registered in the system such as HIS or RIS each time a test is performed on a subject. The registration of the test information into the database 20 may be performed automatically by an apparatus that performs a test (for example, an electrocardiograph that measures an electrocardiogram, a polygraph, or the like) or manually by a user such as a physician or a medical professional.

Examples of the medical data also include medical history. The medical history includes diagnosis results for a disease that a subject is suffering from or a disease that the subject has suffered from in the past, records of treatments that the subject has received, information on physical constitutions such as allergies, and the like. The diagnosis results include information on the position, range, classification, extent, and the like of a disease in addition to the name of the disease. The diagnosis results are not limited to the diagnosis results by a physician, and may also be the result of an automatic diagnosis. Examples of the medical history also include a “diagnostic name” output by an automatic diagnosis algorithm by performing an automatic diagnosis on a subject, and a “treatment candidate” made by a treatment suggestion algorithm based on data obtained from a test and the diagnostic name output by the automatic diagnosis algorithm. The medical history is registered in the system such as HIS or RIS, for example, as a part of an electronic medical record.

The medical data registered in the database 20 are not limited to physiological indicators (biomarkers) as long as the medical data can be used to evaluate the condition of a subject. That is, any data that can be related to the condition of the subject is included in the example of the medical data.

The acquisition function 34a acquires the medical data from the database 20 as illustrated in FIG. 3. For example, the acquisition function 34a acquires the medical data from the database 20 via the network NW. Alternatively, the acquisition function 34a may receive medical data via the input interface 31, or acquire medical data acquired by apparatuses such as the medical image diagnostic apparatus 10 or an electrocardiograph, from the apparatuses. That is, the acquisition function 34a may acquire the medical data without the intervention of the database 20.

The acquisition function 34a acquires various medical data on a plurality of subjects including a subject P. The subject P is a subject to be diagnosed. For example, the acquisition function 34a acquires, from the database 20, subject information, medical images, test results, medical history, and the like associated with a patient ID of the subject P. Similarly, the acquisition function 34a acquires, from the database 20, subject information, medical images, test results, medical history, and the like associated with a patient ID of each subject other than the subject P. The following description is given on the assumption that various medical data on the subject P is referred to as a medical data group A3, and various medical data on a subject other than the subject P is referred to as a medical data group A4. The medical data group A3 is an example of a first medical data group. The medical data group A4 is an example of a second medical data group.

Which subject is to be the subject P to be diagnosed may be determined before the acquisition function 34a acquires the medical data group, or may be determined after the acquisition of the medical data group. For example, the subject P is set in advance according to user input or the like, and the acquisition function 34a acquires medical data groups of a plurality of subjects including the subject P. For example, the acquisition function 34a may acquire a medical data group for arbitrary subjects, and then select the subject P to be diagnosed from the subjects from which the medical data group is acquired.

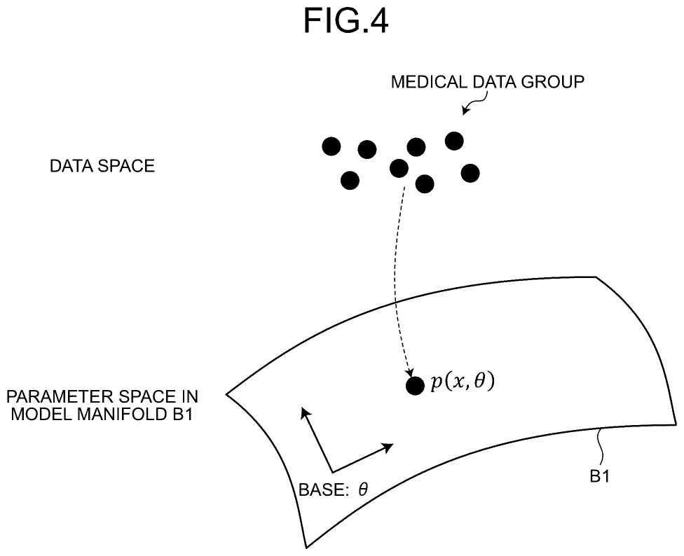

Subsequently, the determining function 34b determines a position in a model manifold by projecting the medical data group onto the model manifold. Specifically, the determining function 34b first determines a statistical model. Subsequently, a model manifold B1 in FIG. 4 is defined according to the statistical model determined by the determining function 34b. The model manifold B1 is, for example, a Riemannian manifold.

For example, the determining function 34b receives an operation of selecting a statistical model from a user via the input interface 31, and determines the statistical model. For example, the determining function 34b automatically determines a statistical model according to the medical data group acquired by the acquisition function 34a. Alternatively, the statistical model may be preset.

A model manifold corresponding to the statistical model determined by the determining function 34b is described below as the model manifold B1. The determining function 34b determines a position in the model manifold B1 by projecting the medical data group onto the model manifold B1. For example, as illustrated in FIG. 4, the determining function 34b assumes that data are distributed according to the statistical model and determines a position p(x, θ) corresponding to the medical data group in a parameter space (parameter space of the model manifold B1) including a certain parameter G.

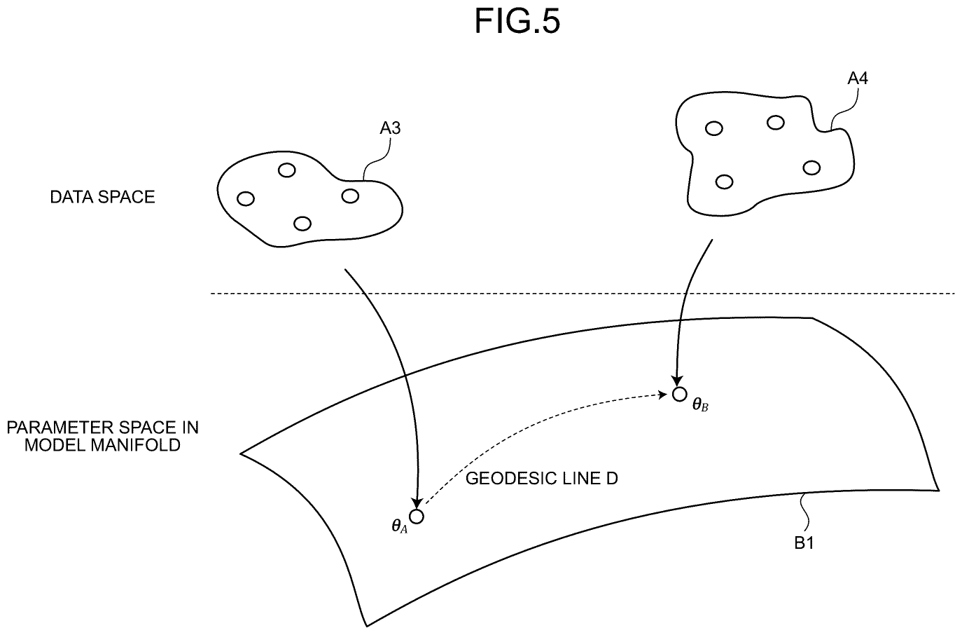

For example, as illustrated in FIG. 5, the determining function 34b determines a position θ_{A} in the model manifold B1 by projecting the medical data group A3 about the subject P onto the model manifold B1. The determining function 34b also determines a position θ_{B} in the model manifold B1 by projecting the medical data group A4 about a subject other than the subject P onto the model manifold B1. That is, the medical data group A3 and the medical data group A4 can be captured as biological manifolds, and the determining function 34b converts each biological manifold into a position (point) in the model manifold B1. The position θ_{A} is an example of the first position. The position θ_{B} is an example of the second position.

Subsequently, the calculation function 34c calculates a geodesic distance (length of a geodesic line D illustrated in FIG. 5) based on the position θ_{A} and the position θ_{B}. That is, the calculation function 34c calculates the distance between the position θ_{A} and the position θ_{B} along the model manifold B1. For example, the calculation function 34c can calculate the geodesic distance according to the following formula (1). In the following formula (1), G represents a Fisher information matrix.

$$D [ {θ}_{A} \vert {θ}_{B} ] = \sum d {θ}^{T} Gd θ \qquad ( 1 )$$

Although FIG. 5 illustrates determining two positions in the model manifold B1, the determining function 34b can determine as many positions in the model manifold B1 as the number of subjects whose medical data are registered in the database 20. The calculation function 34c can calculate as many geodesic distances as the number of combinations of the positions determined in the model manifold B1. However, the calculation function 34c may calculate the geodesic distance only between a position corresponding to the subject P to be diagnosed and a position corresponding to another subject.

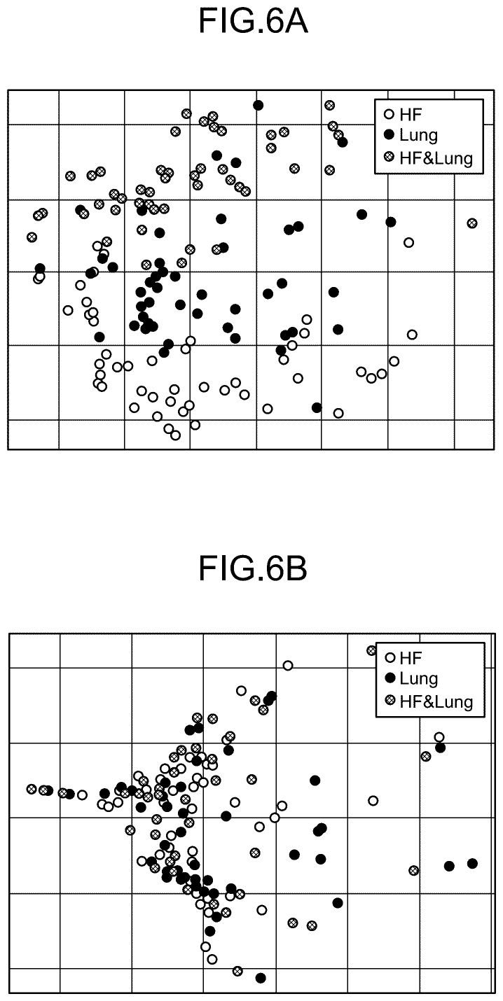

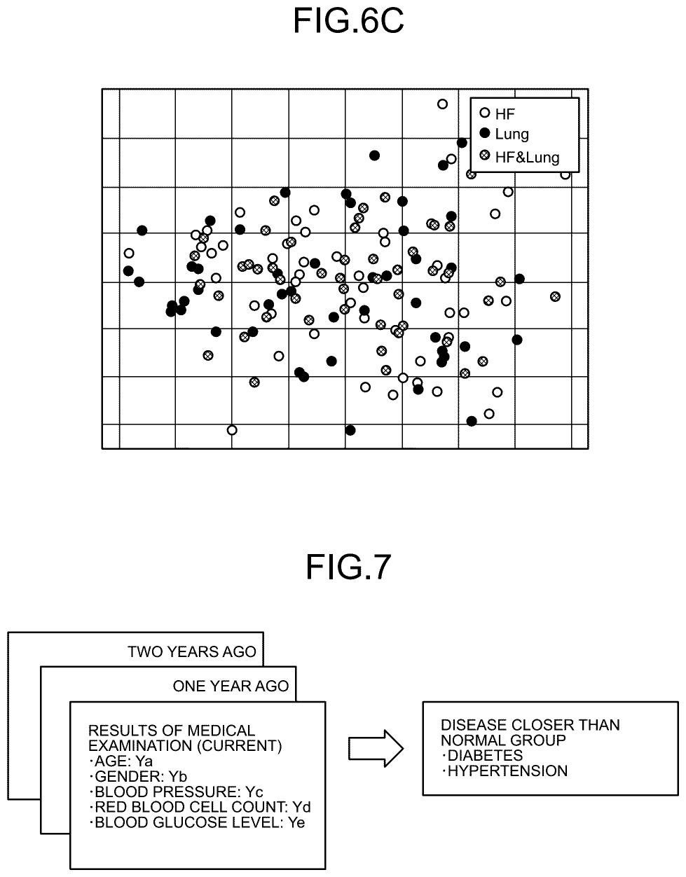

FIG. 6A illustrates an example of geodesic distance calculations for about several tens of subjects. FIG. 6A illustrates a projection of each position determined in the model manifold B1 by the determining function 34b onto a two-dimensional plane so that the magnitude relationship between the geodesic distances calculated in the model manifold B1 is preserved. In FIG. 6A, points corresponding to subjects who are suffering from heart failure are indicated by “HF”, points corresponding to subjects who are suffering from lung cancer are indicated by “Lung”, and points corresponding to subjects who are suffering from both heart failure and lung cancer are indicated by “HF & Lung”.

In FIG. 6A, the points corresponding to “HF & Lung” are concentrated in an upper part, the points corresponding to “Lung” are concentrated in the center, and the points corresponding to “HF” are concentrated in a lower part. That is, geodesic distances between subjects with the same disease are shorter, and geodesic distances between subjects whose diseases do not match are longer. In this way, FIG. 6A illustrates a distribution divided by disease of the subject, and the condition of each subject is represented.

The condition of the subject P can also be estimated by projecting a point corresponding to the subject P to be diagnosed onto the plane in FIG. 6A. For example, when the point corresponding to the subject P is projected to the center in FIG. 6A, it can be estimated that the subject P may have lung cancer.

FIGS. 6B and 6C are illustrated for comparison with FIG. 6A. FIG. 6B illustrates that, for example, as illustrated in FIG. 2, Euclidean distances between medical data groups are calculated in a data space and each medical data group is projected onto a two-dimensional plane so that the magnitude relationship between the calculated distances is preserved. In FIG. 6B, the “HF”, “Lung”, and “HF & Lung” distributions overlap one another. Even though the point corresponding to the subject P is projected onto the plane in FIG. 6B, estimating the condition of the subject P is difficult.

FIG. 6C illustrates that each medical data group is projected onto the model manifold B1, Euclidean distances are calculated in the parameter space of the model manifold B1, and each position projected onto the model manifold B1 is projected onto a two-dimensional plane so that the magnitude relationship between the calculated distances is preserved. In FIG. 6C, although some trends are seen, the “HF”, “Lung”, and “HF & Lung” distributions almost overlap. Even though the condition of the subject P is estimated by projecting the point corresponding to the subject P onto the plane in FIG. 6C, the reliability of the estimation is low.

As described above, FIG. 6A illustrates a clearer representation of the condition of each subject than in FIGS. 6B and 6C. That is, by projecting the medical data group onto the model manifold B1 and determining a position in the model manifold B1 by the process of the determining function 34b and calculating the geodesic distance in the model manifold B1 by the process of the calculation function 34c, information indicating the condition of each subject can be extracted with high accuracy, thereby enabling accurate estimation of the condition of the subject P.

The output function 34d performs output based on the geodetic distance calculated by the calculation function 34c. For example, the output function 34d estimates the condition of the subject P on the basis of the geodesic distance and displays the estimated results as diagnostic support information.

FIG. 7 illustrates an example of display by the output function 34d. In the example illustrated in FIG. 7, the acquisition function 34a acquires the results of current and past (one year ago, two years ago, or the like) medical examinations of the subject P as the first medical data group. The first medical data group includes data such as current and past age, gender, blood pressure, red blood cell count, and blood glucose level. The acquisition function 34a also acquires, as the second medical data group, the results of current and past medical examinations of a plurality of subjects other than the subject P.

The determining function 34b also determines the first position in the model manifold B1 by projecting the first medical data group about the subject P onto the model manifold B1. The determining function 34b also determines a plurality of second positions in the model manifold B1 by projecting each of a plurality of second medical data groups about a plurality of subjects other than the subject P onto the model manifold B1. The calculation function 34c also calculates the geodesic distance between the first position and each of the plurality of second positions.

Subsequently, the output function 34d estimates a disease closer than a normal group for the subject P on the basis of the calculated geodesic distances. That is, on the basis of the geodesic distances, the output function 34d estimates a disease candidate for the subject P. For example, the output function 34d determines a second position having a shorter geodesic distance with the first position about the subject P than a threshold value, and determines a subject corresponding to the determined second position as a similar subject of the subject P. The threshold value for determining the similar subject can be determined by a user, for example. The output function 34d may also automatically set the threshold value on the basis of, for example, a distance to a normal patient (for example, an average value or the like).

Subsequently, the output function 34d determines a disease that the similar subject is suffering from as a disease candidate for the subject P. For example, the output function 34d determines a plurality of similar subjects, and determines diseases that the similar subjects are suffering from at a rate higher than the threshold value, as disease candidates closer than the normal group of the subject P. For example, as illustrated in FIG. 7, the output function 34d displays information on the display 32 as diagnostic support information, the information indicating that the disease candidates for the subject P that are closer than the normal group are “diabetes” and “hypertension”.

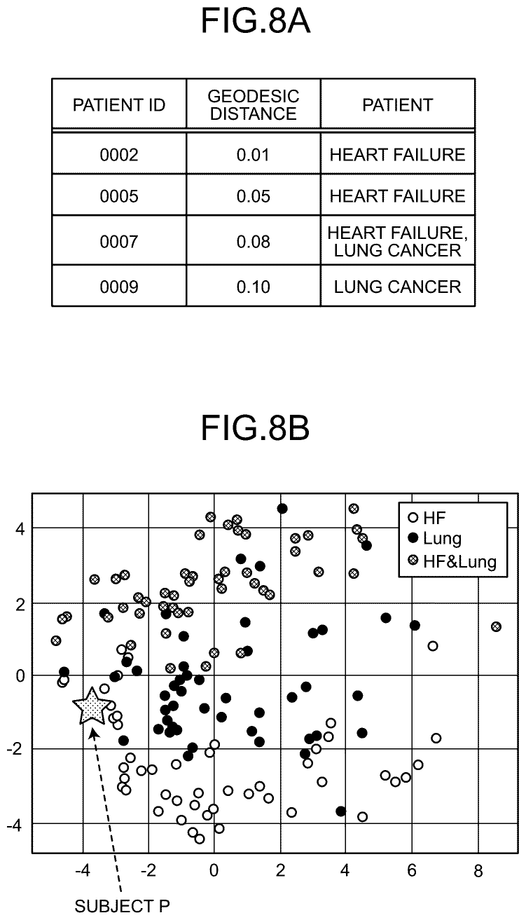

FIG. 8A illustrates another example of display by the output function 34d. The determining function 34b determines the first position and the plurality of second positions in the model manifold B1, like the case of FIG. 7. The calculation function 34c also calculates the geodesic distance between the first position and each of the plurality of second positions.

The output function 34d also determines a second position having a shorter geodesic distance with the first position about the subject P than the threshold value, and determines a subject corresponding to the determined second position as a similar subject of the subject P. For example, the output function 34d determines a subject with a patient ID “0002”, a subject with a patient ID “0005”, a subject with a patient ID “0007”, and a subject with a patient ID “0009” illustrated in FIG. 8A as similar subjects of the subject P.

Subsequently, the output function 34d displays the similar subjects of the determined subject P on the display 32 as diagnostic support information. For example, as illustrated in FIG. 8A, the output function 34d displays patient IDs of the similar subjects, the geodesic distances between the first position about the subject P and the second positions, and diseases that the subjects are suffering from, in association with one another. A user such as a physician referring to the display in FIG. 8A can ascertain that the subject P has “heart failure” and “lung cancer” as diseases closer than the normal group.

The geodesic distances for similar subjects with “heart failure” are “0.01”, “0.05” and “0.08”, respectively, while the geodesic distances for similar subjects with “lung cancer” are “0.08” and “0.10”, respectively. In this way, since the geodesic distances for the similar subjects with “heart failure” are generally short, the user can determine that the subject P may have “heart failure” with a particularly high possibility.

The output function 34d may also display a distribution diagram as illustrated in FIG. 6A as diagnostic support information. That is, the output function 34d may project each position determined in the model manifold B1 onto a two-dimensional plane and display the projected positions on the display 32 as a scatter diagram so that the magnitude relationship between the geodesic distances calculated in the model manifold B1 is preserved. At this time, the output function 34d may indicate the condition of the subject corresponding to each point by the shape and color of each point in the scatter diagram. As illustrated in FIG. 8B, the output function 34d may also highlight a point corresponding to the subject P on the scatter diagram.

The output function 34d may also receive the selection of a point in FIG. 8B and display information on a subject corresponding to the selected point. For example, although the case of displaying information on similar subjects is described in FIG. 8A, the output function 34d may display the information on the subject corresponding to the selected point in FIG. 8B, instead of or in addition to the information on the similar subjects.

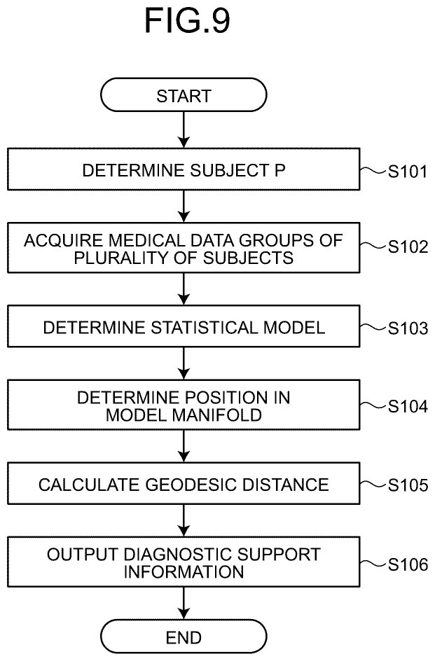

A series of flow of the process performed by the medical information processing apparatus 30 is described below with reference to FIG. 9. FIG. 9 is a flowchart illustrating an example of the process performed by the medical information processing apparatus 30 according to the first embodiment. Steps S101 and S102 are steps implemented by the acquisition function 34a. Steps S103 and S104 are steps implemented by the determining function 34b. Step S105 is a step implemented by the calculation function 34c. Step S106 is a step implemented by the output function 34d.

First, the processing circuitry 34 determines the subject P to be diagnosed (step S101), and acquires medical data groups of a plurality of subjects including the subject P (step S102). Subsequently, the processing circuitry 34 determines a statistical model (step S103) and determines the position of each medical data group in a model manifold associated with the statistical model (step S104). That is, the processing circuitry 34 determines the first position in the model manifold by projecting the first medical data group about the subject P onto the model manifold. The processing circuitry 34 also determines the second position in the model manifold by projecting the second medical data group about subjects other than the subject P onto the model manifold.

Subsequently, the processing circuitry 34 calculates a geodesic distance on the basis of the first position and the second position (step S105). That is, the processing circuitry 34 calculates a distance between the position corresponding to the subject P on the model manifold and each of the positions corresponding to the subjects other than the subject P on the model manifold, along the model manifold. Subsequently, the processing circuitry 34 outputs diagnostic support information based on the geodesic distance (step S106). For example, the processing circuitry 34 performs the displays as illustrated in FIGS. 7, 8A, and 8B.

The flowchart in FIG. 9 is merely an example, and various variations are possible.

For example, determining the subject P at step S101 may be performed after step S102, step S103, or step S104. In this case, the subject P to be diagnosed is selected from subjects subject to the acquisition of the medical data group. Determining the subject P at step S101 may also be performed after step S105. In this case, at step S105, the calculation function 34c calculates a geodesic distance between combinations of arbitrary subjects, and after the subject P is determined, determines the geodesic distance about the subject P.

As described above, the statistical model may be preset. In this case, step S103 can be omitted. The diagnostic support information may be output by other apparatuses different from the medical information processing apparatus 30. For example, the output function 34d may generate the diagnostic support information on the basis of the geodesic distance, and transmit the generated diagnostic support information to other apparatuses via the network NW. Alternatively, the output function 34d may transmit the geodesic distance calculated by the calculation function 34c to other apparatuses via the network NW.

As described above, the acquisition function 34a according to the first embodiment acquires the first medical data group and the second medical data group different from the first medical data group. The determining function 34b determines the first position by projecting the first medical data group onto the model manifold associated with the statistical model, and determines the second position by projecting the second medical data group onto the model manifold. The calculation function 34c also calculates the geodesic distance based on the first position and the second position. This enables the medical information processing apparatus 30 according to the first embodiment to easily provide diagnostic support that comprehensively takes medical data into account.

That is, unlike the case where the diagnostic support information is generated by a machine learning model built to solve a specific task, the medical information processing apparatus 30 allows easy implementation of diagnostic support without the need to prepare tasks and learning data in advance or to perform relearning when data trends or tasks are changed.

According to the medical information processing apparatus 30, unlike the case where a distance between manifolds is calculated in a data space, since representative points need not to be determined for comparison, diagnostic support can be performed by comprehensively taking medical data into account. For example, diagnostic support can be performed by evaluating not only the instantaneous condition of a subject, but also the conditions at a plurality of points in time and changes over time.

For example, the determining function 34b may determine a probability distribution such as a normal distribution, a mixed normal distribution, a binomial distribution, or a Bernoulli distribution as a statistical model, and project a medical data group onto a model manifold associated with the statistical model, thereby determining the first position in the model manifold. That is, the determining function 34b may determine a position corresponding to the medical data group in a parameter space of the model manifold B1 associated with the probability distribution.

When the probability distribution is used as the statistical model, methods for estimating parameters corresponding to the medical data group include maximum likelihood estimation, maximum A posteriori (MAP) estimation, Bayesian estimation, and the like. For example, when a medical data group X_{A} is given, a parameter θ can be estimated by the following formula (2) for maximum likelihood estimation.

$${\hat{θ}}_{A} = arg {max}_{θ} Pr ( {X}_{A} \vert θ ) \qquad ( 2 )$$

For example, the determining function 34b may determine a neural network having any structure as a statistical model and project a medical data group onto a model manifold associated with the statistical model, thereby determining the first position in the model manifold. In this case, the determining function 34b uses a loss function such as least square error or negative log likelihood in order to estimate parameters, and updates the parameters to minimize or maximize a loss function value. The parameters can be updated using any gradient descent method such as stochastic gradient descent (SGD) or ADAM. For example, the parameters can be updated by the following formula (3). In the following formula (3), “α” is a learning rate and “L” indicates the loss function.

$$θ ← θ - α \frac{\partial L}{\partial θ} \qquad ( 3 )$$

For example, the calculation function 34c trains the neural network to minimize or maximize the loss function “L (θ; X_{B})” of a different group of data X_{B} (for example, data included in the medical data group A4) with the position θ_{A} illustrated in FIG. 5 as an initial value. The calculation function 34c calculates a minute change amount “dθ” in the parameter by the gradient descent method. The calculation function 34c also calculates a minute distance “ds” by, for example, the following formula (4). Subsequently, the calculation function 34c repeats the above process until the value of the loss function “L(θ; X_{B})” converges or the determined number of times is reached, and calculates the sum of the minute distances “ds” as the geodesic distance. In the following formula (4), G represents a Fisher information matrix.

$${ds}^{2} = d {θ}^{T} Gd θ \qquad ( 4 )$$

In addition to the embodiments described above, various other variations may be made.

For example, in the embodiments described above, medical data about a certain subject has been described as a medical data group. That is, the medical data group with the subject as a unit has been described. However, the embodiment is not limited thereto.

For example, the acquisition function 34a may acquire a part of the medical data about the certain subject as a medical data group. To give an example, the acquisition function 34a may acquire, as a medical data group, medical data acquired during a period when the subject was a specific age, acquire, as a medical data group, medical data acquired during a period when the subject engaged in specific lifestyle habits, or acquire, as a medical data group, medical data acquired during a period when the subject was suffering from a specific disease.

For example, the acquisition function 34a may also acquire medical data on a plurality of subjects as one medical data group. To give an example, the acquisition function 34a may acquire medical data of a plurality of blood-related subjects as one medical data group, acquire medical data of a plurality of subjects who engage in specific lifestyle habits as one medical data group, or acquire medical data of a plurality of subjects who are suffering from a specific disease as one medical data group.

The displays in FIGS. 7, 8A, and 8B are merely examples, and various variations are possible. For example, as described above, the output function 34d can determine similar subjects of the subject P on the basis of the geodesic distance. On the basis of medical data of the similar subjects, the output function 34d can estimate a temporal transition of the condition of the subject P.

The following describes a case where the subject P is currently “40 years old” and a similar subject is currently “50 years old”. When a geodesic distance based on a first position based on a medical data group including medical data of the subject P of “30 to 40 years old” and a second position based on a medical data group including medical data of a similar subject of “30 to 40 years old” is the shortest, the output function 34d can estimate a temporal transition of the condition of the subject P up to “age 50” on the basis of medical data of the similar subject of “40 to 50 years old”, and output the estimated result as diagnostic support information.

For example, when the condition of the similar subject who is “50 years old” is good, the output function 34d can present, for example, the lifestyle habits and medications taken by the similar subject who is “40 to 50 years old” and the treatment provided to the similar subject as recommended measures to maintain the good condition of the subject P. For example, when the condition of the similar subject who is “50 years old” is not good, the output function 34d can present, for example, regular checkups and early initiation of treatment for a disease as recommended measures.

The term “processor” used in the above description means, for example, circuitry such as a CPU, a graphics processing unit (GPU), an ASIC, or a programmable logic device (for example, a simple programmable logic device (SPLD), a complex programmable logic device (CPLD), and a field programmable gate array (FPGA)). When the processor is, for example, a CPU, the processor reads and executes computer programs stored in storage circuitry to implement functions. On the other hand, when the processor is, for example, an ASIC, the functions are directly incorporated in the circuitry of the processor as logic circuitry instead of storing the computer programs in the storage circuitry. Each processor of the embodiment is not limited to being configured as single piece of circuitry for each processor, and one processor may be configured by combining a plurality of pieces of independent circuitry to implement the functions thereof. The plurality of components in each diagram may be integrated into one processor to implement the functions thereof.

Each component of each apparatus according to the embodiments described above is a functional concept and does not necessarily have to be physically configured as illustrated in the drawings. That is, the specific forms of dispersion or integration of each apparatus is not limited to those illustrated in the drawings, but can be configured by functionally or physically dispersing or integrating all or part thereof in arbitrary units according to various loads and usage conditions. Moreover, each processing function performed by each apparatus can be implemented in whole or in part by a CPU and a computer program that is analyzed and executed by the CPU, or by hardware using wired logic.

The medical information processing method described in the embodiments described above can be implemented by executing a pre-prepared medical information processing program on a computer such as a personal computer or a workstation. The medical information processing program can be distributed via a network such as the Internet. The medical information processing program can also be recorded on a non-transitory recording medium readable by a computer, such as a hard disk, a flexible disk (FD), a CD-ROM, a MO, and a DVD, and executed by being read from the recording medium by a computer.

At least one of the embodiments described above can easily provide diagnostic support that comprehensively takes medical data into account.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
acquire a first medical data group and a second medical data group different from the first medical data group;
determine a first position in a model manifold associated with a statistical model by projecting the first medical data group onto the model manifold, and determine second positions in the model manifold by projecting the second medical data group onto the model manifold; and
calculate a geodesic distance based on the first position and the second position.

2. The medical information processing apparatus according to claim 1, wherein the processing circuitry acquires medical data of a subject to be diagnosed as the first medical data group, and acquires, as the second medical data group, medical data of other subjects different from the subject to be diagnosed.

3. The medical information processing apparatus according to claim 2, wherein the processing circuitry determines a position having the shorter geodesic distance with the first position than a threshold value among a plurality of the second positions respectively corresponding to a plurality of the other subjects, determines the other subject corresponding to the determined position as a similar subject of the subject to be diagnosed, and outputs medical data of the similar subject as diagnostic support information based on the geodesic distance.

4. The medical information processing apparatus according to claim 2, wherein the processing circuitry determines a position having the shorter geodesic distance with the first position than a threshold value among a plurality of the second positions respectively corresponding to a plurality of the other subjects, determines the other subject corresponding to the determined position as a similar subject of the subject to be diagnosed, determines a disease that the similar subject is suffering from as a disease candidate for the subject to be diagnosed, and outputs the disease candidate as diagnostic support information based on the geodesic distance.

5. The medical information processing apparatus according to claim 2, wherein the processing circuitry determines a position having the shorter geodesic distance with the first position than a threshold value among a plurality of the second positions respectively corresponding to a plurality of the other subjects, determines the other subject corresponding to the determined position as a similar subject of the subject to be diagnosed, estimates a temporal transition of a condition of the subject to be diagnosed on the basis of the similar subject, and outputs an estimated result as diagnostic support information based on the geodesic distance.

6. A medical information processing method comprising:
acquiring a first medical data group and a second medical data group different from the first medical data group;
determining a first position in a model manifold associated with a statistical model by projecting the first medical data group onto the model manifold, and determining second positions in the model manifold by projecting the second medical data group onto the model manifold; and
calculating a geodesic distance based on the first position and the second position.

7. A non-transitory computer readable medium comprising instructions that cause a computer to execute processes of:
acquiring a first medical data group and a second medical data group different from the first medical data group;
determining a first position in a model manifold associated with a statistical model by projecting the first medical data group onto the model manifold, and determining second positions in the model manifold by projecting the second medical data group onto the model manifold; and
calculating a geodesic distance based on the first position and the second position.
